(19) United States
(12) Patent Application Publication
(10) Pub. No.: US 2022/0152362 A1
(43) Pub. Date: May 19, 2022

(54) INTRAVASCULAR GAS EXCHANGE DEVICE AND METHOD

(71) Applicant: Agitated Solutions Inc., Oakdale, MN (US)

(72) Inventors: Morgan Evans, Apple Valley, MN (US); Gary Heit, Redwood City, CA (US); Benjamin Arcand, Minneapolis, MN (US); Carl Lance Boling, San Jose, CA (US); Jennifer Chmura, Minneapolis, MN (US); Eric Sabelman, Menlo Park, CT (US); Stephen Ruoss, Redwood City, CA (US)

(73) Assignee: Agitated Solutions Inc., Oakdale, MN (US)

(21) Appl. No.: 17/529,220

(22) Filed: Nov. 17, 2021

Related U.S. Application Data
(60) Provisional application No. 63/133,668, filed on Jan. 4, 2021.
(60) Provisional application No. 63/114,923, filed on Nov. 17, 2020.

(51) Int. Cl. A61M 25/10

(52) U.S. Cl. CPC A61M 25/1002; A61M 2025/1086

(57) ABSTRACT

In some implementations, an intravascular gas exchange catheter includes (a) a catheter wall extending from a proximal end to a distal end; (b) a first internal lumen coupled to a first lumen port at the proximal end and adjacent at least a portion of the catheter wall, and a second internal lumen coupled to a second lumen port at the proximal end; and (c) an interior space enclosed by the catheter wall and disposed at the distal end, wherein the first internal lumen and second interior lumen are fluidly isolated from each other along a length of catheter wall but fluidly coupled to each other at the interior space. The catheter wall may include a porous material that facilitates diffusion of a target gas through the catheter wall, from or to a space exterior to the catheter wall, to or from the first lumen.

### CROSS-REFERENCE TO RELATED APPLICATIONS

This application claims priority to U.S. Patent Application Ser. No. 63/133,668, titled “IVCO2 REMOVAL DEVICE,” filed on Jan. 4, 2021; and to U.S. Patent Application Ser. No. 63/114,923, titled “INTRAVASCULAR GAS EXCHANGE DEVICE AND METHOD,” filed Nov. 17, 2020. This application incorporates the entire contents of the foregoing application herein by reference.

### TECHNICAL FIELD

Various implementations relate generally to intravascular gas exchange.

### BACKGROUND

Lung injury, whether chronic in nature or acute in onset, is a significant clinical problem and the third leading cause of death in the United States. Acute respiratory distress syndrome (ARDS), in particular, has a mortality rate of approximately 45% and affects 190,000 patients annually. More broadly, acute respiratory failure (ARF) affects over 300,000 Americans each year, drastically reducing lung capacity—often to 30% (or less) of normal function.

Conventional treatment for these conditions may include intermittent positive-pressure ventilation—a form of assisted or controlled respiration where oxygen-enriched air is delivered to the lungs under pressure. This treatment can cause oxygen toxicity and pressure injury to the lung tissue, beyond the original injury that precipitated the reduced lung capacity.

In the case of ARDS—typically recognized as severe hypoxemia in patients already critically ill—one of the current ventilation strategies is lung protective ventilation, which in some patients results in severe hypercapnia—resulting in the need for removal of CO₂ from the blood. In acute exacerbations of chronic obstructive pulmonary disease (COPD)—where hospitalization occurs in approximately 700,000 patients annually with a corresponding mortality rate of ˜20%—a device that can temporarily manage CO₂ levels may prevent the need for intubation. Patients with COPD requiring invasive mechanical ventilation have a higher risk of prolonged weaning or failure to wean compared to other causes of acute hypercapnic respiratory failure. A supplemental CO₂ removal device may reduce weaning time and prevent tracheotomy. In addition, pandemics such as H1N1 and Covid-19 can potentially overwhelm the available pool of mechanical ventilators, so alternative lung support devices may provide means to treat patients by being able to maintain these patients with non-invasive ventilation in conjunction with CO₂ removal devices and correspondingly, decreased time on ventilators by shortening weaning times.

Current hypercapnia treatment often involves extracorporeal CO₂ removal (ECCO2R), which requires removing and pumping circulating blood from a large central vein through an artificial lung gas exchange device. Example ECCO2R gas-exchange devices include Hemodec's Decap system, ALung's Hemolung, and Novalung's AVCO2R.

In some cases, removal of carbon dioxide is paired with oxygenation—often referred to extracorporeal membrane oxygenation (ECMO). As with ECCO2R, with ECMO, blood is pumped from a patient's body to an external device that removes carbon dioxide and adds oxygen; then oxygenated blood is returned to the patient's body—thereby providing respiratory support to persons whose lungs are unable to provide adequate gas exchange to sustain life.

Although ECMO and ECCO2R can sustain life for a short period of time for those who are seriously ill, both are associated with numerous high-risk complications—including uncontrollable bleeding, blood clots and stroke, and severe infection, which often result in death. Even with advanced ventilator support and ECMO, ARF proves fatal for approximately 50% of patients, with some age groups experiencing mortality as high as 60%. Furthermore, ECMO can add additional functional complexity to patient care, as such systems often require dedicated personnel for use (perfusion technologist) and involve significant extracorpreal tubing runs and connections. These all provide potential sites for clot formation and also increase the expense of intensive care unit (ICU) management due to the additional complexity and personal need for safe ECMO procedures. ECCO2R devices are often associated with complications, including device-related pump, oxygenator and heat-exchanger malfunction, air embolism, coagulation factor depletion, and clot formation. In addition, patients have experienced hemolysis, anticoagulation-related bleeding, and catheter site bleeding, kinking, infection, and occlusion.

Some efforts have been made to make intravenous gas exchange devices. Among those devices, CardioPulmonics' intravenacaval gas exchange device (IVOX) is believed to be the only respiratory-assist device to date to undergo phase I and II human clinical trials. The IVOX device demonstrated some removal of CO₂ and a measurable reduction in ventilator requirements in normocapnia. Ultimately, however, the benefit did not outweigh poor hemodynamic tolerance, incidence of mechanical/performance failures, and its catheter insertion size of 34 French requiring a specialized surgeon. Other attempts to replace ECMO, which have not progressed as far as the IVOX, have been made—including the “Hattler” device, the Internal Impeller Respiratory Assist Catheter (IPRAC) and the “HIMOX” device—all of which, including IVOX, employ a large number hollow fiber membranes (HFMs) to perform gas exchange.

While hollow fiber membranes (HFMs) are commonly employed in extravascular circuits due to their high surface area (lower volume of blood needed, lower resistance to blood flow), incorporating them intravascularly does not work well. The aforementioned devices failed for a variety of reasons, including, in many cases, excessive blood flow resistance, active mixing causing vascular wall damage, excessive catheter insertion size, lower basal exchange than expected, and thrombus formation. In addition, computational modeling and experiments have shown that the effective surface area of exchange of HFMs is smaller than expected in high flow environments like the inferior vena cava (IVC); and spacing between HFMs may be necessary to prevent boundary layer formation, which can severely limit gas exchange.

Some progress has been made in the understanding of how to provide effective ventilation of patients with acute lung injuries; however, there remains a need for improved ventilator strategies and sustainable alternatives to ECMO and ECCO2R in the treatment of ARF and ARDS, and in current ventilation management practices to decrease the incidence of fatality.

### SUMMARY

Described herein are devices and methods that avoid pitfalls of extravascular circuits and employ unique approaches to solve the “boundary layer” problem. Some implementations effectively leverage bioactive CO₂ enzymes, flow rates, and sweep gas parameters. Some implementations employ membranes folded into fins and arranged radially about a central catheter. Some implementations employ other features (e.g., membrane, geometry, sweep gas) to optimize CO₂ extraction. Some implementations can be deployed using widely known Seldinger techniques. Some implementations have a sufficiently small form factor to be clinically and commercially viable.

Also disclosed herein are various implementations of an intravascular gas exchange catheter that can be temporarily implanted in a patient's circulatory system to assist in oxygenating the patient's blood and/or in removing carbon dioxide (e.g., as either bicarbonate form or as a dissolved gas) from a patient's blood. In some implementations, such a device can be employed to assist in resolving hypoxemia and/or hypercapnia; with each variable being controlled independently. Various implementations may be implanted similarly to a peripherally inserted central catheter or a central line.

### BRIEF DESCRIPTION OF THE DRAWINGS

FIG. 1A illustrates an exemplary intravascular gas exchange catheter (IGEC).

FIG. 1B illustrates an exemplary radial cross-section of the IGEC of FIG. 1A.

FIG. 1C illustrates an exemplary longitudinal cross-section of the IGEC of FIG. 1A.

FIG. 1D depicts the diffusion of gas into the IGEC of FIG. 1A, in one implementation.

FIG. 2A illustrates another exemplary IGEC.

FIG. 2B illustrates an exemplary radial cross-section of the IGEC of FIG. 2A, according to one implementation.

FIGS. 2C-2E illustrates exemplary radial cross-sections of the IGEC of FIG. 2A, according to other implementations.

FIG. 2F illustrates an exemplary radial cross-section of the inflatable balloon structure shown in FIG. 2A, according to one implementation.

FIG. 2G illustrates a surface of an exemplary portion of an inflatable balloon structure, according to one implementation.

FIG. 2H illustrates an exemplary radial cross-section of the inflatable balloon structure shown in FIG. 2A, according to another implementation.

FIG. 2I illustrates a manner in which wings or petals of an inflatable balloon structure may be disposed on an IGEC wall, in some implementations.

FIG. 2J illustrates an exemplary segment of an IGEC with nested spiral wings.

FIG. 2K illustrates exemplary adjacent segments of an IGEC with nested spiral wings.

FIG. 3A illustrates another exemplary IGEC.

FIG. 3B illustrates exemplary functional detail of the IGEC of FIG. 3A.

FIG. 3C illustrates sensors, valves and controllers that may be employed with an IGEC.

FIG. 3D illustrates an exemplary three-lumen IGEC.

FIG. 4A illustrates various aspects of a human circulatory system.

FIG. 4B depicts one possible arrangement of an exemplary IGEC in a patient.

FIG. 4C depicts another possible arrangement of an exemplary IGEC in a patient.

FIG. 4D depicts another possible arrangement of an exemplary IGEC in a patient.

FIG. 4E depicts another possible arrangement of complimentary IGECs in a patient.

FIG. 5A depicts an exemplary intravenous CO₂ removal (IVCO2) device in a patient's inferior vena cava.

FIG. 5B illustrates detail of a single fin of the exemplary IVCO2 device shown in FIG. 5A.

FIG. 6A is a top view of three stacked modules in an exemplary IVCO2 device, showing a staggered-fin arrangement.

FIG. 6B is a perspective view of a distal end of an exemplary IVCO2 device having nine fins.

FIG. 7 illustrates a benchtop circuit model used to test various aspects of an IVCO2 membrane.

### DETAILED DESCRIPTION

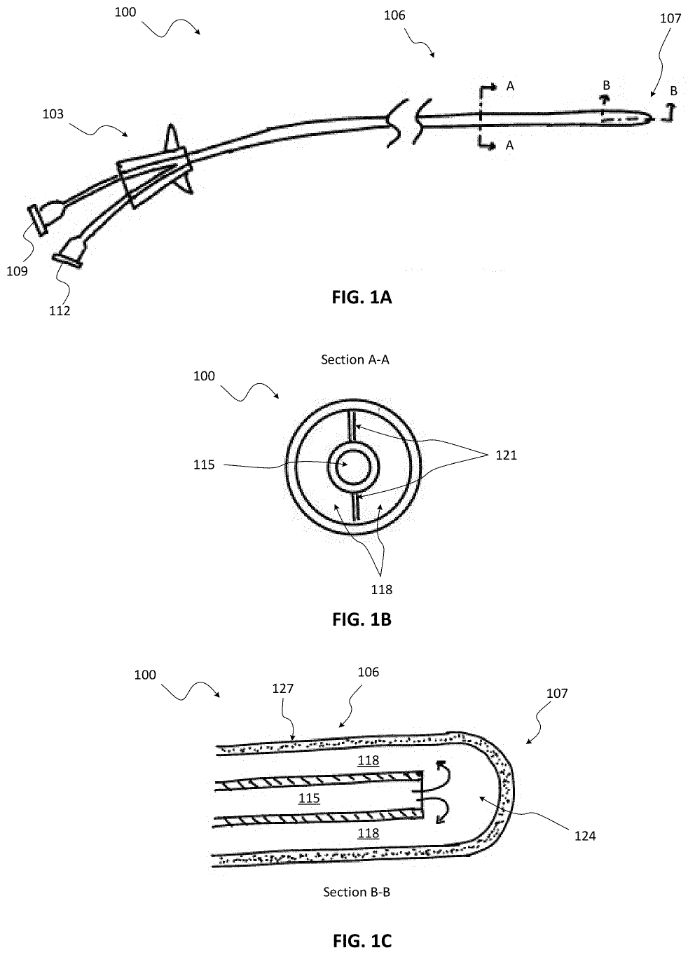

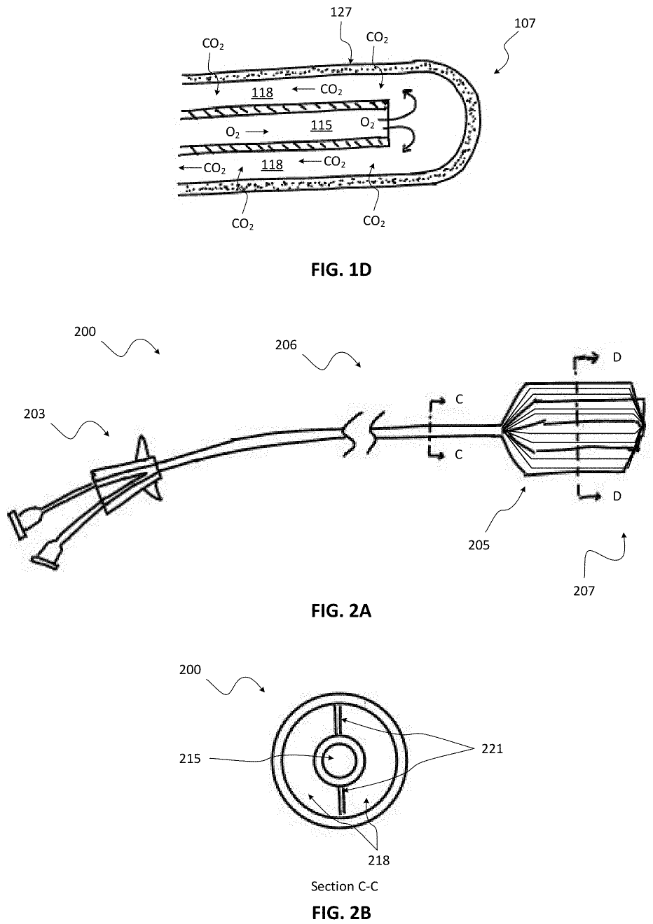

FIG. 1A illustrates an exemplary intravascular gas exchange catheter (IGEC) 100. In some implementations, the IGEC 100 could be temporarily inserted into the vasculature of a patient suffering from hypercapnia, whose normal respiratory function may be compromised, to intravascularly remove excess carbon dioxide. More specifically, as will be described with reference to subsequent figures, an outer wall of the portion of the IGEC 100 that is temporarily inserted to the vasculature of a patient may be porous to carbon dioxide (e.g., be configured to facilitate diffusion or passage of carbon dioxide from blood adjacent the IGEC 100 into the IGEC 100 itself), and the IGEC 100 may be configured to remove carbon dioxide that flows into the IGEC 100 to assist in resolving the patient's hypercapnia.

In the implementation shown, the IGEC 100 is a two-lumen device, configured similarly to a peripherally inserted central catheter (a PICC line) or a central line. That is, the IGEC 100 has a proximal portion 103 that, in use, remains outside a patient's body; and a distal portion 106 that is configured to be temporarily disposed in a patient's circulatory system. The proximal portion 103 is shown to include a first port 109 and a second port 112, each of which can be fluidly coupled to a different internal lumen.

FIG. 1B illustrates an exemplary radial cross-section of the IGEC 100 shown in FIG. 1A. As shown, the IGEC 100 includes a central lumen 115 and an annular outer lumen 118. One or more webs 121 may also be provided to maintain substantially uniform spacing between the central lumen 115 and the outer lumen 118. FIG. 1B is only exemplary; many other lumen arrangements are possible.

FIG. 1C illustrates an exemplary longitudinal cross-section of the IGEC 100 at its distal tip 107. In some implementations, the outer wall 127 of the distal portion 106 is porous to, or enables diffusion or passage through, of certain gases, such as oxygen and carbon dioxide. As shown, the central lumen 115 terminates prior to the distal tip 107, leaving an interior space 124 for gas flowing from the proximal end 103—for example, through the central lumen 115—to exit the central lumen 115 and return via the outer lumen 118. In some implementations, the central lumen 115 and outer lumen 118 are fluidly isolated from each other along the length of the IGEC 100, except at the interior space 124.

In use in a patient's circulatory system, as depicted in FIG. 1D, some implementations may facilitate removal of carbon dioxide from the blood—specifically by allowing carbon dioxide to diffuse through the outer wall 127 into the outer lumen 118, where, flow of a “sweep” gas from the distal tip 107 to the proximal portion 103 causes removal, intravascularly, of the diffused carbon dioxide.

In some implementations, the sweep gas is oxygen. In such implementations, some oxygen may diffuse out of the IGEC 100, from the outer lumen 118 into the patient's blood stream. In other implementations, the sweep gas is a different gas, such as, for example, nitrogen, helium, hydrogen, or a gas mixture like the atmosphere, containing gases such as nitrogen, oxygen, and hydrogen (including, for example, purified ambient room air). In some implementations, instead of a sweep gas employed, or beside deployment of a sweep gas, a liquid such as lactic acid or glucose may be infused temporarily to promote localized acidification of the blood. In still other implementations, a sweep liquid or gas may include perfluorocarbons or other substances that have a high carbon dioxide solubility.

Regardless of the specific sweep gas employed, the pressure of that sweep gas may be set to promote maximum diffusion of carbon dioxide into the IGEC 100 (and, in some implementations, to promote diffusion of oxygen out of the IGEC 100). That is, the sweep gas pressure may typically be set to a pressure that is lower than the partial pressure of carbon dioxide in the venous blood of a target patient. For example, in some implementations, the sweep gas pressure is set to 2-6 mmH₂O (millimeters of water). In some implementations, the sweep gas will be set to less than 8-12 mmH₂O; in some implementations, the pressure may be 4-6 mmH₂O; and in some implementations, the pressure will preferably be set to 5-6 mmH₂O. In some implementations, the sweep gas pressure may be oscillated between these values. In some implementations, the sweep gas pressure may be modulated by applying a vacuum to one or more of the internal lumens.

The foregoing description is directed to removing, by diffusion, carbon dioxide. In some implementations, other gases, fluids or compounds may also be targeted for removal; and the porous outer wall 127 and the pressure of the sweep gas may be set accordingly. For example, some implementations may target removal of carbonic acid from blood adjacent the IGEC 100; other implementations may target removal of bicarbonate ions from blood adjacent the IGEC 100. In some implementations, a sweep fluid, such as a saline or other ionized solution, may replace a sweep gas. In some implementations, the porous outer wall 127 may be doped with a material that facilitates carbon dioxide diffusion and removal (e.g., a carbonic anhydrouser). In some implementations, rather the outer wall may include non-porous membranes that facilitate a first stage of permeation or diffusion, followed by a second stage where diffused compounds are removed.

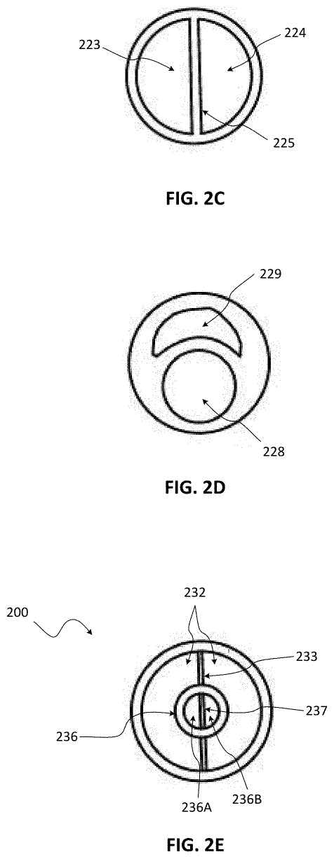

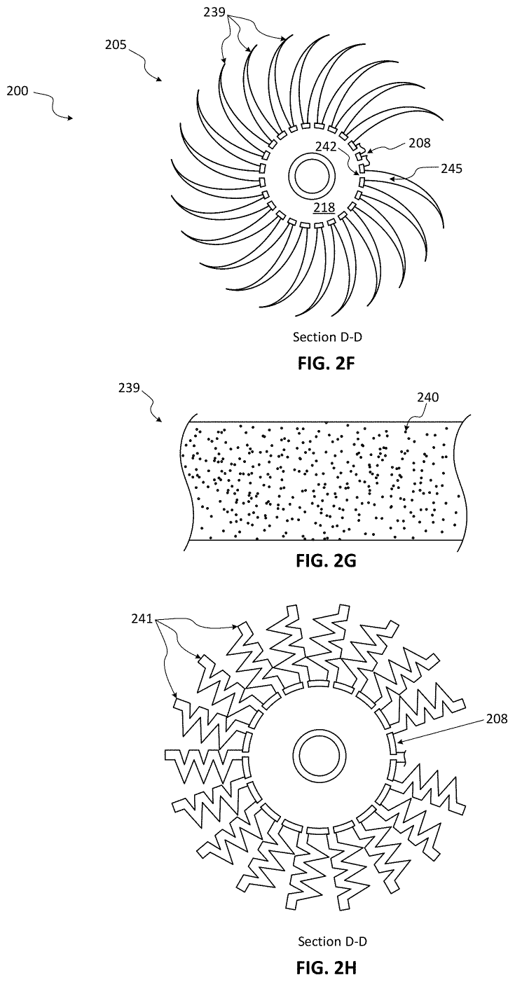

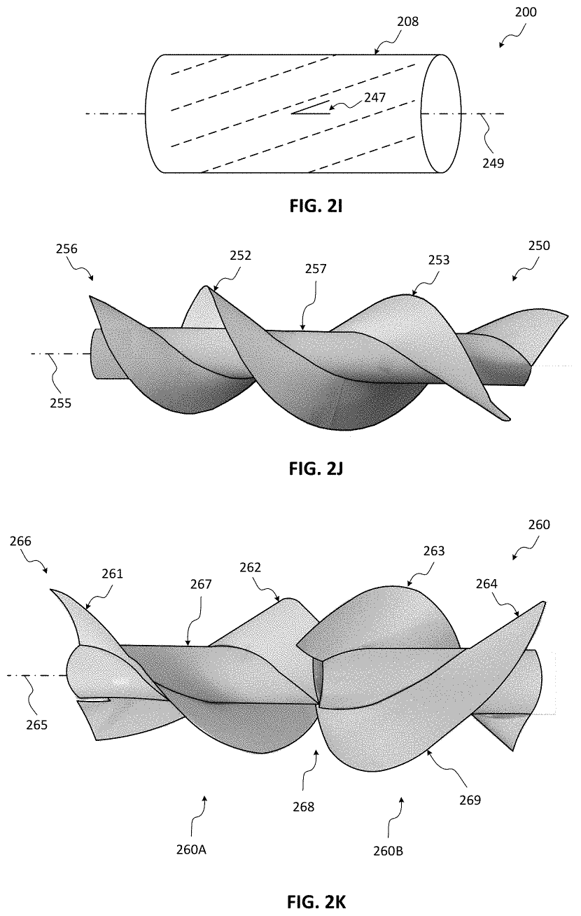

FIG. 2A illustrates another exemplary IGEC 200. In some implementations, the IGEC 200 could be temporarily inserted into the vasculature of a patient suffering from hypoxemia, whose normal respiratory function may be compromised, to intravascularly oxygenate the patient's blood stream. More specifically, as will be described with reference to subsequent figures, a portion of the IGEC 200 that is temporarily inserted to the vasculature of a patient may be porous to oxygen (e.g., be configured to facilitate release of oxygen from inside the IGEC 200 into blood adjacent the IGEC 200), and the IGEC 200 may be configured to intravascularly oxygenate a patient's blood to assist in resolving the patient's hypoxemia.

As shown, the exemplary IGEC 200 is a two-lumen device having a proximal portion 203 configured to remain outside of a patient, and a distal portion 206 configured to be temporarily disposed in a patient's circulatory system. The IGEC 200 includes an inflatable balloon structure 205 at its distal tip 207. The inflatable balloon structure 205 is shown as inflated, but the reader will appreciate that that inflatable balloon structure 205 would be implanted in a patient in a deflated configuration and with a retractable introducer sheath (not shown in FIG. 2A).

FIG. 2B illustrates a radial cross-section taken along section lines C-C of the IGEC 200 shown in FIG. 2A, in one implementation. As with the IGEC 100, whose radial cross-section is illustrated in FIG. 1B, the IGEC 200 includes a central lumen 215 and an annular outer lumen 218. One or more webs 221 may be provided to maintain substantially uniform spacing between the central lumen 215 and the outer annular lumen 218.

Other implementations are possible. For example, as shown in FIG. 2C, a first lumen 223 and a second lumen 224 may be separated from each other by a central wall 225. In another implementation, as shown in FIG. 2D, a larger circular lumen 228 may be provided, as well as a smaller semi-circular lumen 229. In still other implementations, as depicted in FIG. 2E, the IGEC 200 may include a large annular lumen 232, which may be bisected by one or more web structures 233; and a central lumen 236 that also may be bisected by one or more web structures 237. In some implementations, a web structure 237 may completely bisect the central lumen 236 to form two parallel central lumens 236A and 236B; in some implementations, the outer lumen 232 may also be completely bisected by web structures 233.

FIG. 2F is a radial cross-section along the section lines D-D shown in FIG. 2A, according to one implementation. As shown in this implementation, the inflatable balloon structure 205 includes a plurality of wings 239, and each wing may be anchored to the outer wall 208 of the IGEC 200, facilitating inflation of each wing 239). Such implementations may facilitate flow of blood over a greater surface area than would be possible relative to a nearly cylindrical balloon structure; and this greater surface area may promote more gas exchange than would otherwise be possible.

The surface of each wing 239 may be perforated with a plurality of apertures 240, as depicted in FIG. 2G; and the apertures 240 may be configured to facilitate gas communication from inside the IGEC 200 to outside the IGEC 200 (e.g., to a patient's blood flowing past the wings 239). Passages 242 (see FIG. 2F) may be provided to fluidly couple lumen 218 with an interior space 245 formed by the surface of each wing 239, to enable flow of gas from the interior space 245, out of each wing 239, into a patient's adjacent blood stream.

In some implementations, each wing 239 is configured to extend radially outward (e.g., inflate) when a pressure inside the lumen 218 and interior space 245 is positive; and further configured to collapse onto an outer wall of the IGEC 200 when a pressure inside the lumen 218 and interior space 245 is not positive (e.g., negative or zero).

In other implementations, each wing 239 is configured to automatically expand, for example, after removal or retraction of a delivery sheath (not shown). For example, the wing 239 may include an internal strut system made of a shape-memory material, such as nitinol, that automatically returns to an expanded shape upon removal of the sheath.

Internal features may be provided to facilitate flow of gas, even in cases where the wings 239 are only partially expanded. For example, internal surface treatment or structures may create internal passages through which gas flow is possible, regardless of the state of deployment or expansion of the wings 239.

External features (not shown) may also be provided to prevent wings 239 from sticking to each other, or at least to minimize fibrin or platelet sticking. For example, surfaces of the wings 239 may be coated in a manner that facilitates uninterrupted flow of blood between adjacent wings. As another example, surface treatments may be provided to create physical spaces or interstices between wings 239, even when such wings 239 are adjacent each other.

In some implementations, a surface of the wings 239 is made of a flexible or semi-flexible membrane, such as, for example, polyurethane, silicone, or polyether block amides (e.g., PEBAX™). In other implementations, the wings 239 may be a less compliant material, such as, for example, polyester, nylon or nitinol. In some implementations, edges of the wings 239 are rounded to minimize trauma to adjacent blood vessels.

Apertures 240 on the surface of the wings 239 may be formed by laser drilling, laser cutting, or in another manner. In some implementations, the apertures are between ½ um (500 Angstroms) and about 4 um and are configured to facilitate creation of microbubbles having diameters of about 1-10 um in adjacent blood.

In some implementations, pleated “petals” 241 may be employed in place of the wings 239, as illustrated in FIG. 2H. Like the wings 239, such petals 241 may be configured to extend radially outward (e.g., inflate) when a pressure inside the lumen 218 is positive; and further configured to collapse onto an outer wall 208 of the IGEC 200 when a pressure inside the lumen 218 is not positive (e.g., negative or zero). As mentioned above, unfurling may also be accomplished not by pressure but with an endoskeleton or scaffolding made from a memory material that expands (e.g., upon release of an introducer or delivery sheath). The petals 241 may also be supported by a “cage” (not shown) that contacts the vascular wall either periodically or continuously to hold the IGEC in place and/or center the petals 241.

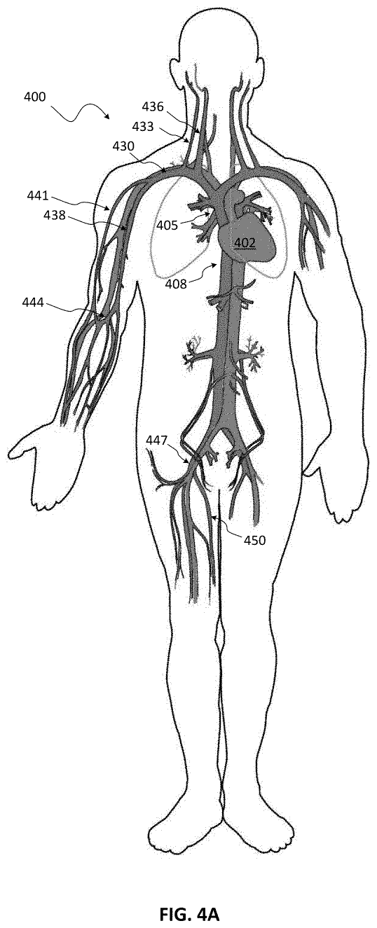

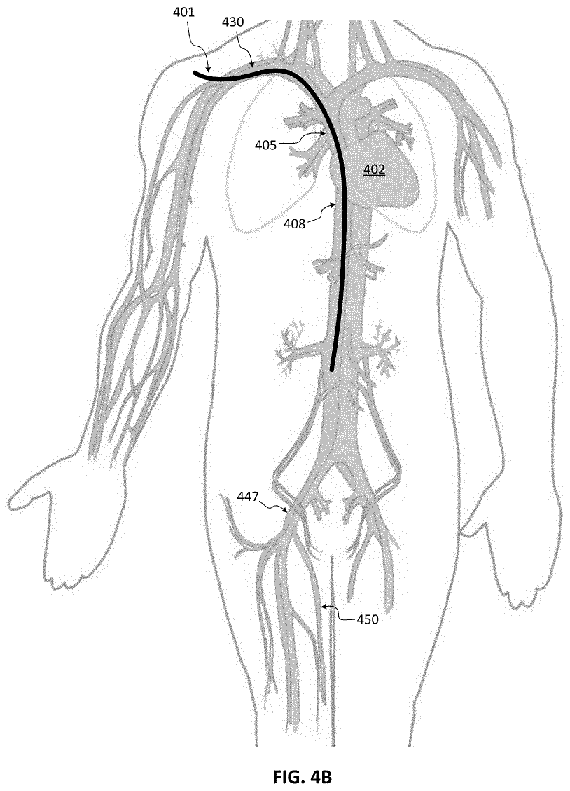

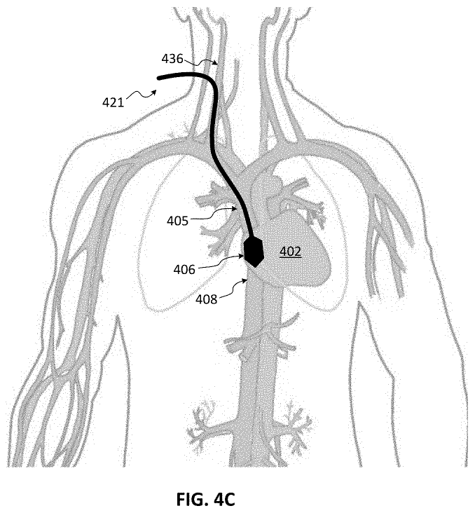

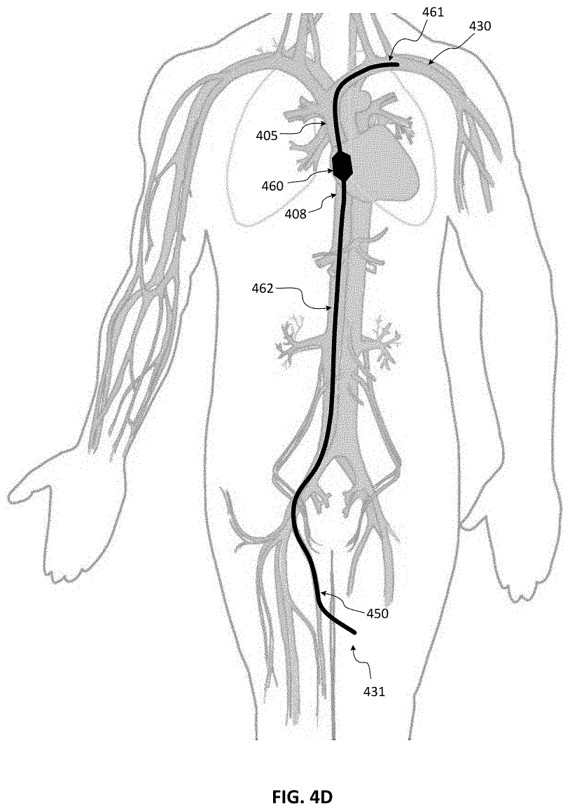

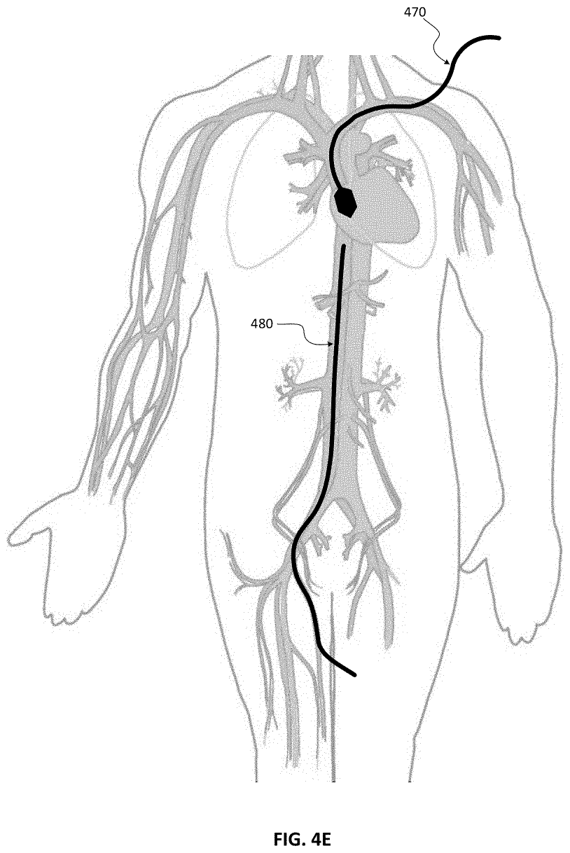

The petals 241 or wings 239 may be initially collapsed when the IGEC 200 is initially implanted in a patient, and they may be expanded or inflated only when they are properly positioned intravascularly (e.g., at the superior vena cava, inferior vena cava, or atrium, in some implementations, as described with reference to FIG. 4B and FIG. 4C). Moreover, the petals 241 or wings 239 may be configured to collapse when the IGEC 200 is withdrawn from the patient (e.g., back through an introducer sheath (not shown)).

To facilitate collapse onto the wall 208 of the IGEC 200 when the IGEC 200 is withdrawn from a patient, the petals 241 or wings 239 may be attached to the outer wall 208 of the IGEC at an angle 247 relative to an axis 249 of the IGEC 200, as is illustrated in FIG. 2I. With this arrangement, it may be possible to twist the IGEC 200 slightly as it is withdrawn into a sheath, so as to facilitate collapse of the petals 241 or wings 239 in a manner that prevents their interference with each other or with the sheath itself. In some implementations, edges of the petals 241 or 239 may also be tapered to further facilitate orderly collapse and retraction into a sheath.

FIG. 2J illustrates one segment 250 of an IGEC that comprises a first spiral wing 252, and a second spiral wing 253 nested within the first spiral wing 252. As shown, the spiral wings 252 and 253 are disposed at an angle relative to an axis 255 of the IGEC 250, and the “twist” of the nested spiral wings 252 and 253 is to the right, when viewed from the left end 256 of the IGEC 250. Such an implementation may cause blood flowing past the segment 250 to flow around a central shaft 257 of the IGEC in a circular direction. Such a circular flow may cause greater contact with surfaces of the wings 252 and 253, which may, in turn, result in a greater degree of gas exchange between the flowing blood and interior of the segment 250.

In cases where the segment 250 is a portion of an IGEC that is configured to deliver oxygen to the blood, more oxygen may be so delivered, because of this increased flow or more turbulent flow. That is, more blood may come in contact with the wings 252 and 253; and the flow or turbulence itself may dislodge more microbubbles of oxygen as they are formed than may otherwise be dislodged with a different geometry.

In cases where segment 250 is a portion of an IGEC that is configured to extract carbon dioxide from the blood, the increased flow or more turbulence may have a similar effect on blood-IGEC gas exchange, but in the opposite direction. That is, more carbon dioxide may be extracted from the blood because of increased blood-IGEC contact facilitated by the specific geometry.

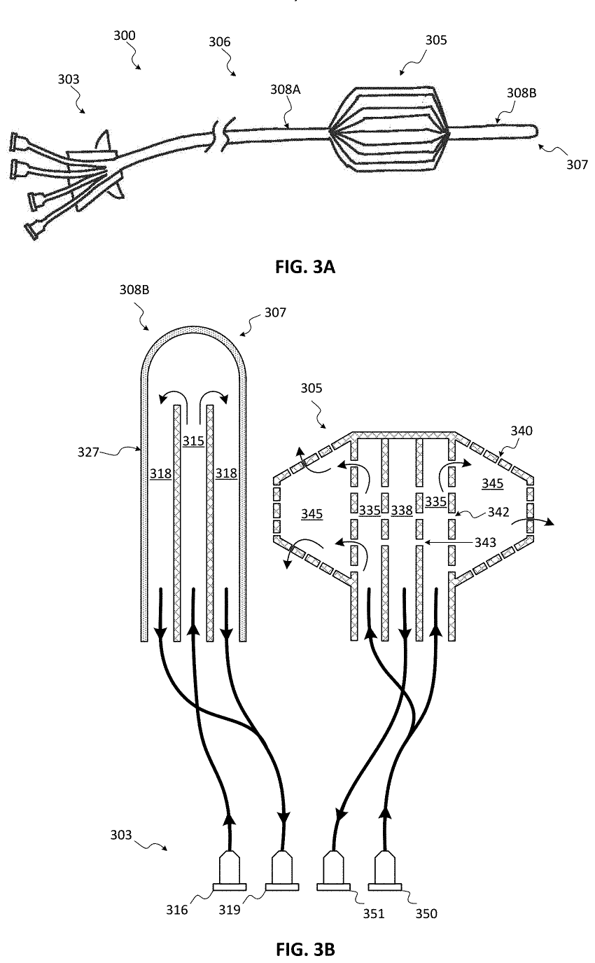

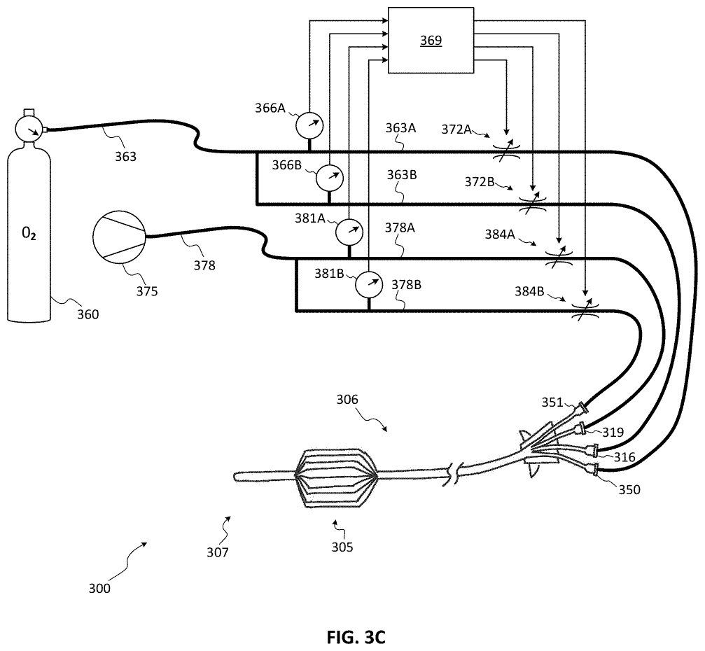

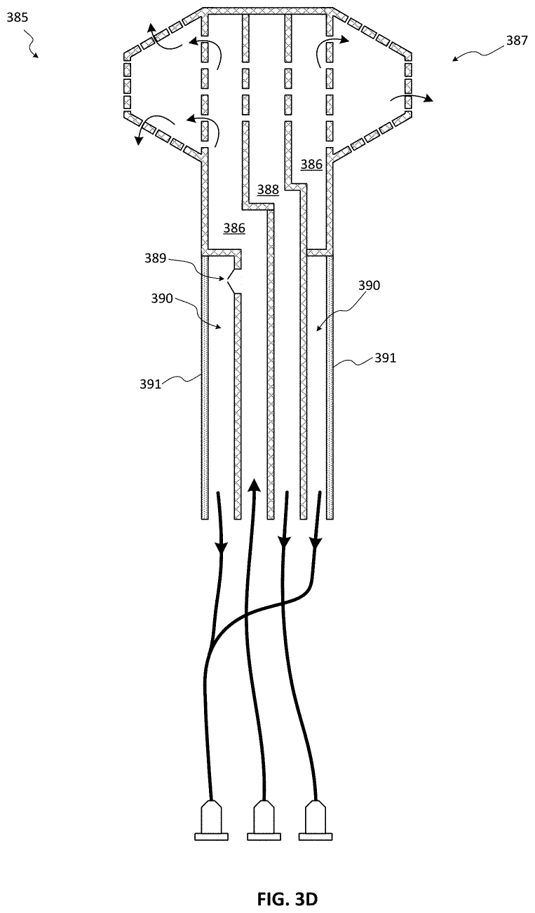

In some implementations, a structure such as that shown in FIG. 2J may be employed with other structures described and illustrated herein. For example, in some implementations, the segment 250 may be employed along a length of an IGEC that is dedicated to removal of carbon dioxide, up to a separate balloon structure (not shown) that is configured to oxygenate blood (e.g., a segment 250 with spiral wings 252 and 253 may extend along an entire length of the distal segments 308A and 308B shown in FIG. 3A). In such implementations, the increased flow or turbulence may not only promote enhanced gas exchange along the segment 250 itself, but such increased flow or turbulence may promote enhanced gas exchange at the separate balloon structure (e.g., by dislodging additional microbubbles of oxygen than may otherwise be dislodged).

In some implementations, further turbulence may be induced by disposing segments of spiral wings in opposite directions. For example, as shown in FIG. 2K, a segment 260 may include two sub-segments: a sub-segment 260A with spiral wings 261 and 262 that are disposed in a clockwise direction relative to an axis 265 of a central shaft 267 of the IGEC, when viewed from the left side 266 of the segment 260; and a sub-segment 260B with spiral wings 263 and 264 that are disposed in a counterclockwise direction relative to the same axis 265 and reference point 266. At the interface 268 of the two sub-segments 260A and 260B, the different directions of the various spiral wings 261, 262, 263 and 264 may create additional turbulence in blood flowing past these spiral wings. This additional turbulence may further disrupt a boundary between the blood and surfaces of the wings 261, 262, 263 and 264 in a manner that facilitates additional blood-IGEC gas exchange.

In some implementations, sub-segments 260A and 260B are repeated along a significant length of an IGEC (e.g., along distal segments 308A and 308B shown in FIG. 3A) in a manner that substantially increases surface area that is available for blood-IGEC gas exchange while at the same time directing blood flow in a manner that creates turbulence and otherwise disrupts a boundary layer at the blood-IGEC interface in a manner that promotes enhanced gas exchange.

In addition enhancing blood-IGEC gas exchange, implementations such as those depicted in FIG. 2J and FIG. 2K may have other advantages. In particular, relative to other geometries, nested spirals may inherently minimize damage to vessel walls. A “leading edge” of each spiral (e.g., leading edge 269 in FIG. 2K; generally, the outermost edge, relative to a central shaft—at which one “wall” of the nested spiral meets an opposing wall) is generally parallel to the wall of a vessel through which it passes, which may minimize trauma to the endothelium and intima of the vessel. In addition to being generally parallel to the vessel wall, the angle of the spiral walls themselves may promote their folding or partially collapsing as the IGEC is advanced through a blood vessel—further reducing risk of trauma to the endothelium and intima.

FIG. 3A illustrates another exemplary IGEC 300. In some implementations, the IGEC 300 could be temporarily inserted into the vasculature of a patient whose normal respiratory function has been compromised, and who may be suffering both hypercapnia and hypoxemia. That is, as will be described with reference to subsequent figures, an outer wall of a portion of the IGEC 300 may be porous to carbon dioxide (e.g., be configured to facilitate diffusion of carbon dioxide from blood adjacent the IGEC 300 into the IGEC 300 itself), and another portion of the IGEC 300 may be porous to oxygen (e.g., be configured to facilitate release of oxygen from inside the IGEC 300 into blood adjacent the IGEC 300)—such that the IGEC 300 is configured to intravascularly oxygenate a patient's blood to assist in resolving the patient's hypoxemia, and remove carbon dioxide from the patient's blood to assist in resolving the patient's hypercapnia.

As shown, the IGEC 300 is a four-lumen device having a proximal portion 303 configured to remain outside of a patient, and a distal portion 306 configured to be temporarily disposed in a patient's circulatory system. The IGEC 300 includes an inflatable balloon structure 305 between its proximal portion 303 and a distal tip 307, a distal segment 308A on one side of the balloon structure 305 and a second distal segment 308B on the other side of the balloon structure 305. In some implementations, the distal segments 308A and 308B are porous to carbon dioxide, and the balloon structure 305 is porous to oxygen.

FIG. 3B illustrates exemplary functional inner detail of the segment 308B and the balloon structure 305, in one implementation. As shown, an inner lumen 315 is coupled to a first lumen port 316 at the proximal portion 303 of the IGEC 300; and an outer lumen 318 is coupled to a second lumen port 319 at the proximal portion 303 of the IGEC 300. In some implementations, the first lumen port 316 and corresponding inner lumen 315 carry a sweep gas to the distal tip 307, where the sweep gas exits the inner lumen 315 and returns to the proximal portion 303 via the outer lumen 318 and corresponding second lumen port 319. An outer wall 327 may be porous to certain gases or compounds (e.g., carbon dioxide, carbonic acid, bicarbonate ions, etc.), allowing such gases or compounds to diffuse from blood adjacent the segment 308B, through the porous outer wall 327, into the outer lumen 318. The flow of sweep gas through the outer lumen 318 may cause removal of the diffused gases or compounds, and this removal (and the corresponding change in concentration and/or partial pressure differentials of such gases or compounds on either side of the porous wall 327) may facilitate additional diffusion into the outer lumen 318. Through this process, carbon dioxide, for example, may be removed from a patient's bloodstream intravascularly.

Though not separately depicted in FIG. 3B, the segment 308A may have a similar structure as segment 308B. That is, the segment 308A may share an inner lumen 315 and outer lumen 318 structure with the segment 308B, also be fluidly coupled to the lumen ports 316 and 319, and also have a porous outer wall 327—such that gases or compounds can be removed from both segment 308B and 308A.

As shown, the balloon structure 305 includes a lumen 335 that may be configured to fluidly couple to lumen port 350. In some implementations, the lumen port 350 and corresponding lumen 335 delivers oxygen to the balloon structure 305. The oxygen may be pressurized to facilitate its flow through passages 342; into interior spaces 345 (e.g., within a cylindrical inflated balloon structure 305, or within wings or petals, like those depicted in FIG. 2F and FIG. 2H); and out of the balloon structure 305 through apertures 340. Through the flow of oxygen along this route, microbubbles may be formed in a patient's blood that is adjacent the balloon structure 305; and these microbubbles may oxygenate the patient's blood (e.g., to assist in resolving hypoxemia in the patient).

In some implementations, an additional lumen 338 may be provided and coupled to a lumen port 351. At the balloon structure 305, the lumen 338 may be fluidly coupled to the lumen 335 and the interior space 345 (e.g., through passages 342); and the lumen 338 may serve as a safety feature to facilitate rapid evacuation of oxygen flowing to the balloon structure 305 through the lumen 335, in the event of a rupture or other failure of the lumen 335 or the balloon structure 305. Such a safety feature may reduce the risk of an air embolism from being introduced in a patient in the event of a device failure.

In some implementations, the lumen 338 and corresponding lumen port 351 are omitted, and safety of the overall IGEC 300 may be provided by safety valves or other mechanisms that regulate flow of gases. In other implementations, the lumen 338 and corresponding lumen port 351 are provided, along with safety valves and controllers, exemplary versions of which are now described with reference to FIG. 3C.

FIG. 3C depicts an exemplary system of sensors or pressure gauges, valves and a controller that may be part of the IGEC 300, in some implementations. As shown, the IGEC 300 includes an oxygen source 360 and an oxygen supply line 363. In some implementations, the oxygen supply line 363 divides into two separate oxygen supply lines 363A and 363B. In such implementations, one oxygen supply line 363A may provide oxygen to the balloon structure 305; and another oxygen supply line 363B may provide oxygen as a sweep gas. In such implementations, the pressure of oxygen in the supply line 363A may be greater than the pressure of oxygen in the supply line 363B.

In other implementations, only a single oxygen supply line 363A is provided, and this line may provide both oxygen for the balloon structure 305 and as a sweep gas. In such implementations, a restrictor may be integrated internally to the IGEC 300 (e.g., near the balloon structure 305) to allow oxygen (e.g., at a possibly lower relative pressure than that of the oxygen directed to the balloon structure) to flow to the distal dip 307 and return via an outer lumen to sweep away, for example, carbon dioxide that diffuses into the IGEC 300.

As depicted in FIG. 3C, pressure of the supply line 363A is monitored by a pressure sensor 366A. An output of the pressure sensor 366A is coupled as an input to a controller 369. Upon receipt of a signal from the pressure sensor 366A, the controller 369 may output a control signal to the adjustable valve 372A, to facilitate control of pressure in the supply line 363A. If present, supply line 363B may also include a pressure sensor 366B and a corresponding adjustable valve 372B. With this arrangement, the controller 369 can control pressure of oxygen to the balloon structure 305 (e.g., for intravascular oxygenation) and pressure of oxygen that may be used as a sweep gas. This arrangement is exemplary. In other implementations, a separate gas or fluid source may be employed as a sweep gas or fluid, but the reader will appreciate that a similar control system may be employed.

A vacuum source 375 may also be provided and coupled to the IGEC 300 via a vacuum line 378. In some implementations, the vacuum line 378 is divided into a vacuum line 378A and a vacuum line 378B. As with the oxygen supply lines 363A and 363B, each vacuum line 378A and 378B may include a corresponding pressure sensor 381A or 381B, whose output may be routed to the controller 369. Based on this input, the controller 369 may control corresponding adjustable valves 384A and 384B. In some implementations, one controller may be employed for the vacuum lines 378A and 378B, and a separate controller may be employed for the oxygen supply lines 363A and 363B. In other implementations, such as the one depicted in FIG. 3C, a common controller 369 is employed.

Regardless of the precise architecture, the controller 369 can control the adjustable valves for oxygen supply and vacuum line(s) to maintain safe and effective operation of the IGEC 300. For example, a sudden pressure drop on an oxygen supply line may indicate a rupture of the balloon structure 305 or a component or lumen thereof; and upon detection of such a pressure drop, the controller 369 may cause oxygen supply to be cut off (e.g., by closing valves 372A and/or 372B) and may further increase the vacuum for a period of time (e.g., by temporarily opening valves 384A and/or 384B).

Other sensors may provide input to the controller 369. For example, in some implementations, oxygen and/or carbon dioxide sensors (not shown) may be disposed on the distal portion 306 of the IGEC 300. Such an oxygen sensor that is disposed upstream of the balloon structure 305 may provide an indication of venous blood oxygenation. If this venous blood oxygenation is lower than expected, even with supplemental oxygenation being provided by the IGEC 300, the controller 369 may increase the pressure of the oxygen supply line 363A (e.g., by causing the valve 372A to incrementally open).

As another example, a carbon dioxide, carbonic acid or bicarbonate ion sensor may be provided; and if such a sensor detects higher-than-desired parameters, even with supplemental removal of such gases or substances by the IGEC 300, the controller 369 may adjust appropriate valves 372A, 372B, 384A or 384B to facilitate increased removal of the target substance or gas. In some cases, this may include lowering a sweep gas pressure to promote increased diffusion into the IGEC 300. In other cases, this may include increasing a return vacuum. In still other cases, both sweep gas pressure and vacuum line pressure may be adjusted.

In the implementation shown in FIG. 3C, multiple supply lumens 363A and 363B and multiple vacuum lines 378A and 378B may provide precise control of various parameters. Other implementations may include greater or fewer supply and vacuum lines and lumens. In particular, some implementations may include two oxygen supply lines and one vacuum line, and some implementations may include only a single oxygen line and a single vacuum line. In some implementations, to simplify the internal structure, dedicated lumens may be provided for sweep gas and vacuum lines that are routed to the distal tip 307—separate from lumens for sweep gas and vacuum lines that are routed to a segment of the IGEC 300 that is proximal to the balloon structure 305 (e.g., a six-lumen system). The reader will appreciate that numerous variations are possible.

FIG. 3D illustrates one additional such variation—specifically, a three-lumen IGEC 385. The three-lumen IGEC 385 may include a high-pressure oxygen deliver lumen 386 that is coupled to a balloon structure 387. A high-pressure return lumen 388 may also be included and may, in operation, be coupled to a vacuum pump. With such a configuration, risk of rupture of the balloon structure 387 or release of an air embolism may be minimized—specifically by facilitating rapid evacuation of the IGEC 385 in the event of a device failure. The IGEC 385 may also include a reducer valve 389 that allows fluid communication between the high-pressure supply lumen 386 and an outer circular lumen 390 (e.g., one that may be configured to extract carbon dioxide from a patient's blood, for example, through a porous outer membrane 391). With such a configuration, the high-pressure lumen 386 may supply oxygen that can both oxygenate a patient's blood through the balloon structure 387 and serve as a lower pressure sweep gas through the return lumen 390.

Pressure of the sweep gas in the return lumen 390 may be controlled through design of the reducer valve 389. In some implementations, the pressure drop across the valve 389 is fixed; in other implementations, the pressure drop may be controlled—for example, through an actuator (not shown in detail, but could be a piezoelectric actuator that is controlled by electrical conductors that are integral to the IGEC 385).

FIG. 4A illustrates various aspects of a patient's circulatory system 400 into which an exemplary IGEC may be deployed. At its core is the heart 402, and a system of arteries that extend from the heart and veins that return to the heart. Blood is returned to the heart 402 from throughout the body by the vena cava, which is divided into the superior vena cava 405, which collects blood from the upper portion of the body, and the inferior vena cava 408, which collects blood from the lower portion of the body. Blood flows through the superior vena cava 405 and inferior cava 408 on its way to the right atrium.

The superior vena cava 405 may be accessed through the subclavian vein 430, the external jugular vein 433, the internal jugular vein 436, or from a smaller upstream vein, such as the axillary vein 438, the cephalic vein 441, or the cubital vein 444. The inferior vena cava 408 may typically be accessed via the femoral vein 447 or the saphenous vein 450.

FIG. 4B depicts one possible arrangement of an exemplary IGEC 401. In the implementation shown, the IGEC 401 may be configured to intravascularly remove carbon dioxide from a patient's bloodstream (e.g., as described with reference to FIG. 1A and following). As shown, the IGEC 401 is implanted in the patient through the subclavian vein 430 and extends through the superior vena cava 405 and inferior vena cava 408. In this arrangement, blood returning to the heart 402 of the patient from both upper and lower extremities flows past the IGEC 401, and carbon dioxide in that returning blood may diffuse into the IGEC 401 for intravascular removal.

To increase the surface area of contact between returning blood and the IGEC 401, the length of the IGEC 401 may be even longer than shown. For example, in some implementations, the IGEC 401 may extend to the femoral vein 447 of the patient, to the saphenous vein 450, or beyond. In general, the maximum length of an implanted IGEC 401 may be constrained primarily by its diameter and the corresponding diameters of the vessels in which it is implanted.

In FIG. 4B, the IGEC 401 is depicted as entering the patient through the subclavian vein 430. The reader will appreciate that other entry points are possible. For example, the IGEC 401 may also be implanted through the internal jugular vein 436 (see FIG. 4A), the external jugular vein 433, the axillary vein 438, the cubital vein 444, the femoral vein 447, the saphenous vein 450, other suitable veins (or arteries) in a patient's vasculature. In general, the IGEC 401 may be configured to be implanted in a manner similar to a PICC line or central line.

FIG. 4C depicts a possible arrangement of another exemplary IGEC 421. In the implementation shown, the IGEC 421 may be configured to intravascularly oxygenate a patient's bloodstream (e.g., as described with reference to FIG. 2A and following). As shown, the IGEC 421 is implanted in the patient through the internal jugular vein 436 and extends through the superior vena cava 405, to the right atrium of the patient's heart 402. With this disposition, the IGEC 421 may facilitate oxygenation of blood immediately prior to it being pumped to the lungs of the patient. Moreover, by disposing the balloon structure 406 of the IGEC 421 in the right atrium, maximum space may be provided for the balloon structure 406 to expand, and thus the surface area of the balloon structure 406 through which oxygen is released may be maximized. The balloon structure 406 may also be disposed in the superior vena cava 405 or the inferior vena cava 408, or in both, on either side of their junction at the right atrium.

As with the exemplary IGEC 401 of FIG. 4B, the IGEC 421 shown in FIG. 4C may be implanted through other pathways through the patient's vasculature. For example, the IGEC 401 may be implanted through the patient's external jugular vein 433 (see FIG. 4A), the subclavian vein 430, the axillary vein 438, the cubital vein 444, the femoral vein 447, the saphenous vein 450, other veins (or arteries) in a patient's vasculature that are suitable for receiving a PICC line or central line.

FIG. 4D depicts a possible arrangement of another exemplary IGEC 431. In the implementation shown, the IGEC 431 may be configured to both intravascularly oxygenate a patient's bloodstream and, simultaneously, remove carbon dioxide from the patient's bloodstream (e.g., described with reference to FIG. 3A and following). As shown, the IGEC 431 is implanted in the patient through the saphenous vein 450 and extends through the inferior vena cava 408 and superior vena cava 405, and a distal segment 461 extends up into the patient's subclavian vein. In this exemplary position, a lengthy proximal (relative to the balloon structure 460) segment 462 is positioned to remove carbon dioxide from blood returning to the heart 402 from the lower extremities, and the distal segment 461 is positioned to remove carbon dioxide from blood returning to the heart from the brain (a significant source of the body's carbon dioxide) and upper extremities. Between the proximal segment 462 and distal segment 461 is the balloon structure 460, at the right atrium of the heart 402, where blood can be oxygenated prior to being pumped to the lungs.

As with the previous examples, other methods and locations of implant may be employed. For example, the IGEC 431 could be implanted from the internal jugular vein 436 (see FIG. 4A) and could employ a relatively longer distal segment 461 to extend through the inferior vena cava 408 and beyond. Other arrangements are possible, as facilitated by the diameter of the IGEC 431 and the diameters of the vessels through which the IGEC 431 is disposed.

FIG. 4E depicts a possible arrangement of two separate IGECs operating to both oxygenate a patient's blood and remove carbon dioxide from the patient's blood. As shown, a first IGEC 470 is disposed through the patient's subclavian vein, to the right atrium, where it oxygenates blood in the manner described herein. As shown, a second IGEC 480 is disposed through the patient's saphenous vein and extends to the inferior vena cava. The second IGEC 480 may be configured to remove carbon dioxide from the patient's blood.

In some implementations, operation of the first IGEC 470 and the second IGEC 480 may be coordinated. For example, a common control system, such as that illustrated in and described with reference to FIG. 3C may be employed to control both IGEC 470 and IGEC 480. In other implementations, IGEC 470 and IGEC 480 may be independently controlled.

In some implementations, employing dedicated IGECs for either oxygenation or carbon dioxide removal may enable each IGEC to have a smaller diameter than may otherwise be possible. In such implementations, it may be possible to deploy the IGEC devices from more peripheral veins or deploy such devices in smaller and/or younger patients.

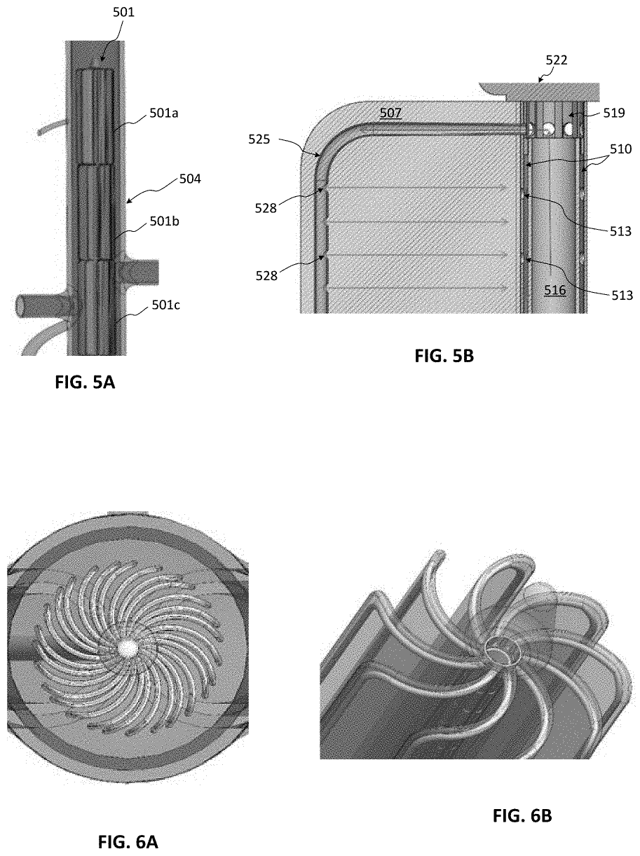

FIG. 5A illustrates an exemplary implementation of an intravenous CO₂ removal (IVCO2R) device 501. In some implementations, the IVCO2R device 501 is inserted through a pateint's femoral vein and guided into the patient's IVC 504 and placed at the right atrium. After implant, placement may involve withdrawing an outer sheath (e.g., consisting, in some implementations, of a braid-reinforced, fluoroethylenepropylene (FEP)-lined Nylon-12 tube) to unfurl the membrane modules. The IVCO2R device 501 may be temporarily implanted (e.g., for 30 days or less) and may then be retrieved through a snare operated by a proximal handle.

In some implementations, three “modules” 501a, 501b and 501c may be stacked and staggered. Some such implementations may have a total membrane surface area of 0.027 m². Each module, in some implementations, may consist of a thin, gas permeable, flat membrane 507 (see FIG. 5B) containing nine fins of 1 mm width when inflated, 5 cm length along the IVC, and 1.75 cm radial length arranged in a turbine pattern around a cannula. The membrane 507 may have an inner structural porous polypropylene layer and an outer, blood-compatible nonporous silicone layer, and may be compliant to reduce the possibility for vessel wall damage. The outer membrane may be coated with carbonic anhydrase (CA) to further facilitate and enhance extraction of CO₂.

A sweep gas may flow through cannulas and membranes, as depicted in FIG. 5B. In the implementation shown, an outer cannula 510 with perforations 513 along its length is disposed at the base of each fin, as well as an inner cannula 516 which terminates into a chamber 519 isolated by a cap 522. Oxygen (or other sweep gas) may be injected through the inner cannula 516 and may reach the distal end of the device, flowing into an isolated chamber 519 separated by the cap 522. This chamber 519 may be connected to tubing lines corresponding to each fin (including the tubing line 525), which may both form the shape of the fins and allow passage of oxygen into the most peripheral portion of the corresponding fin. Suction applied to the outer cannula 510 may pull oxygen (or other sweep gas) radially across the fins and through perforations 528 in the tubing line 525 and perforations 513 in the outer cannula 510, allowing CO₂ to diffuse through the membrane 507 before being returned proximally under suction.

Safety features of the exemplary IVCO2R 501 may include a bleeder valve between the supply and suction lines (not shown), so that when a pressure differential is sensed, suction can be increased to aspirate blood and prevent gas emboli formation. An external controller may monitor the differential pressure across the inlet/outlet gas and flow rate of the oxygen and vacuum, to modulate the sweep gas flow. In addition, the sweep gas flow may be pulsated (e.g., at a rate up to 7 Hz) to promote active mixing between the sweep gas and to-be-extracted CO₂.

In some implementations, the exemplary IVCO2R device 501 uses a smooth, continuous membrane 507 for improved hemocompatibility and smaller device insertion size. (A functional surface area of 0.027 m² is equivalent to 38 HFs of 1.5 mm in diameter—which is smaller in overall surface area than prior HFM respiratory assist catheters.) In many implementations, smaller insertion sizes translate to reduced need for anticoagulative therapy, reduced bleeding, and reduced use/need for blood products during a corresponding procedure.

In some implementations, accelerated diffusion across a membrane may be achieved by catalyzing dehydration of bicarbonate to gaseous CO₂ using CA—an enzyme present on endothelial surfaces of the lungs (CO₂+H₂O↔HCO₃⁻+H⁺). The IVCO2R membrane may be made bioactive in this manner.

FIG. 6B illustrates a turbine shape with nine fins to optimize surface area of blood flow contact while minimizing blood flow obstruction (e.g., <25% of the vessel lumen for safety, in some implementations). With blood in the vena cava moving at a rate of 1-2 L/min, the device is exposed to a much higher flow and thus greater volume of cardiac output than many ECCO2R devices.

Passive mixing may be achieved by an IVCO2R in two ways: (1) creating vortexes around the stator twisted turbine fins and (2) modular stacking of the turbines, like an array of windmill farms. The enhanced hydrodynamic conditions may include both secondary flows and radial mixing. Based on computational fluid dynamic (CFD) modeling, Applicant found that this IVCO2R design has a reasonable balance between lowering the blood path width for CO₂ extraction promotion and ensuring that this is hemodynamically well tolerated. In particular, CFD modeling showed that, in one implementation, an exemplary device would have marginal effect on blood flow dynamics during inspiration. During expiration, the central area of the device may slow blood velocity down by ˜22% while the outer edges may facilitate maintenance of a higher velocity. Velocity decrease near the center may indicate an improved blood flow path for CO₂ extraction without causing a shunt.

Sweep gas may move radially across the fins, starting from the outside of the fin and moving inward. In some implementations, a modular construction provides fresh O₂ sweep gas to each module, effectively implementing counterflow gas exchange. Sweep gas may be exhausted from the fins under vacuum—causing CO₂ that diffuses through the membrane and into the fin to be quickly evacuated and providing a fresh volume of O₂ for subsequent CO₂ removal.

Active mixing may be achieved by pulsating the gas pressure inside the membrane. In some implementations, when pressure is increased, the membrane flexes outward and moves into the low-velocity blood of the boundary layer; when pressure is decreased, the membrane deflates and draws high-velocity blood into the boundary layer. Because blood viscosity can damp movement of the whole vane at high pulsation frequencies, imparting low (˜0.5 Hz) frequency pulsation can cause fins to change their radius of curvature and sweep through the local bloodstream. The combination of high and low frequency pulsation can be varied in real-time according to patient-specific respiratory needs.

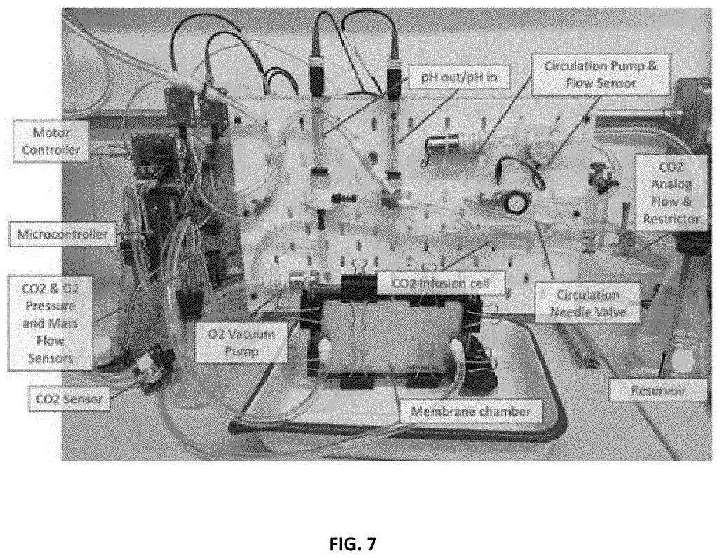

Using the benchtop circuit model shown in FIG. 7, a 2D silicone membrane of surface area 0.01029 m² was tested. On a water flow membrane side, CO₂ was injected through an infusion cell until a steady-state PaCO₂ of 60 mmHg was reached to mimic hypercapnic conditions. Pure O₂ sweep gas under suction was then swept across the membrane at a flow rate of 110 mL/min. The CO₂ removal reached a maximum value of 4.5 mL/min, which, when scaled up to an exemplary IVCO2R membrane size, corresponds to a 35% basal CO₂ removal for an average adult at rest (estimate 250 mL/min CO₂ production). A 9.5% improvement of CO₂ extraction was apparent when increasing the pulsation to 100 Hz.

Static mixing may be further enhanced by staggered fin positions of adjacent modules (see FIG. 5A; see also FIG. 6A, showing three stacked modules, each with nine fins, where the fins in one module are offset relative to the fins of an adjacent module). Additional means for passive control of the blood flow path may include (a) imparting a helical twist to the fins, (b) reversing the curvature from clockwise to counterclockwise in adjacent modules, and (3) varying the resting-state curvature of leading and trailing edges of the fins.

In some implementations, to manufacture an exemplary IVCO2R device, the membrane may require successive folding and heating on metal fixtures to conform to the final, “impeller” shape while reducing internal stresses that may cause the membrane to rupture. The oxygen (or other sweep gas) inlet tubing may have holes cut along its length and may be inserted into a mandrel. A long, rectangular flat membrane may be placed over the folds/valleys of the mandrel on a single plane, and the edges of the fins may be heat sealed around the oxygen inlet tubing. The oxygen inlet tubing may be supported by an internal stainless steel wire spring, which, in some implementations, sets a zero-pressure curvature of the fin and allows the fins to be folded into a compressed shape for insertion and to spring outwards at deployment to their final configuration. The membrane may then be coated with CA by means of a silane bonding agent.

An outer sheath may be manufactured by, for example, sandwiching stainless-steel braid between an FEP liner and Nylon-6 tubing onto a mandrel. The outer sheath may then be reflowed in an oven. A dual lumen catheter may be made by laser cutting perforations in outer tubing and placing inner tubing, cut to length, within. Proximal ends may be temporary coupled to a Duette Silicone, 2-Way Foley Catheter (16 Fr) for connection to the sweep lines. Cap and connector modules of the device may be manufactured and sealed to the inner cannulas.

To integrate various components, the membrane may be wrapped and aligned on the outer cannula, so that the holes in the cannula are aligned with each of the nine fins. The ends of the membrane sheet may be sealed by a lap joint at the cannula-membrane interface. The cap, connected to the inner cannula, may be fed through the top of the assembly. The oxygen inlet tubing may be pushed into respective holes of the cap so that it is within the oxygen chamber (see FIG. 5B). Potential leaks in the seal between the cannula and membrane may be sealed using ultrasonic welding. Additional modules may be similarly connected, using a connector piece that creates an oxygen chamber instead of a cap between modules. The above-described process completes assembly of the distal end. Modules may be tested for leaks and to confirm working pressure within specifications.

At the proximal end, the outer sheath may be placed over the assembly and bonded to a Tuohy-Borst adapter so that it can be locked into place. In some implementations, this can prevent premature deployment. The oxygen and vacuum tubes may be split within a hub for respective connections to the controller.

In some implementations, a controller consists of hookups to an oxygen source tank and to a vacuum pump, as well as to the catheter gas lines. Gas flow meters and pressure sensors may be provided as inputs. The controller may modulate the pressure and flow of the oxygen and the vacuum to inflate and deflate the fins (e.g., at rates up to 7 cycles/sec). Detection of transients in the differential pressure between the inlet/outlet gas lines may cause a safety control shut-off of the inlet oxygen so that blood is aspirated out and gas emboli are prevented in the case of device failure. To assist in withdrawing the device into the outer sheath after treatment is concluded, the controller may only apply vacuum, causing the fins to contract.

Many other variations are possible, and modifications may be made to adapt a particular situation or material to the teachings provided herein without departing from the essential scope thereof. For example, fewer or greater numbers of lumens may be employed; lumens may have different configurations and shapes; the catheters described may include other common features such as guide wires, guide sheaths, introducer sheaths, etc.; access may be provided through other portions of a patient's vasculature than those described; the catheters described may be employed outside of a patient's circulatory system (e.g., in a patient's digestive system, lymphatic system, cranium, respiratory system, etc.); gases, fluids or substances—other than oxygen or carbon dioxide—may be added or removed; flow may be reversed through various cannulas (e.g., the sweep gas may flow from a central cannula to an outer edge of a fin; sweep gas may flow through an inner cannula or outer cannula); a sweep gas other than oxygen may be used; other manufacturing methods than those described may be employed; etc.

To improve oxygenation, some implementations may incorporate mechanisms to agitate the balloon structure, including, for example piezoelectric transducers, ultrasound transducers or mechanical agitators that may be pneumatically powered by incoming gas flow. In some implementations, such mechanisms may produce low frequency agitation (e.g., 1-500 Hz); in other implementations, high frequency agitation may be provided.

Specific structural elements may further improve oxygenation. For example, holes through which oxygen is released may be straight, have a partial exterior bevel, have rounded lips, or have a full conical design geometry. Specific design tradeoffs may be made to improve bubble detachment and homogeneity. In some implementations, the balloon structure may be configured with a geometry similar to a stent, where its level of dilated expansion also determines the size of the holes through which oxygen passes.

Other variations are possible to overcome effects of boundary-layer gas exchange stasis. These variations include wing designs in a corkscrew or alternating left/right helical configurations to promote mild turbulent flow, mechanical agitation via low frequency (e.g., 1-500 Hz) piezoelectric transducers, mechanical rattlers powered by the high-pressure oxygen side, single or double reed shaker values powered either by the sweep gas or high pressure supply side. In some implementations, the boundary layer may also be disturbed by having a double-layered outer wall, where the first layer promotes permeation of bicarb (e.g., through a doped layer) and a second layer promoting diffusion or separation of the bicarb.

Either oxygenation or carbon dioxide removal elements may be constructed such that they rotate or reciprocate to detach microbubbles, or disrupt the blocking boundary layer for gas extraction, respectively. Such rotation or reciprocation could be powered by piezoelectric motors, small DC motors, mechanical vanes in the high-pressure gas side, etc. Such implementations may include additional seals and points that specifically facilitate rotational motion.

Although many of the implementations described may be directed to use in a hospital or ICU setting, other implementations may be configured for long-term remote or at-home use. For example, an IGEC such as the one depicted in FIG. 4D may be implanted in a patient and coupled to a control system such as the one depicted in FIG. 3C—in a form factor that is similar to a left ventricular assist device (LVAD). An oxygen supply may be a semi-portable tank, or a portable oxygen concentrator may be employed. Sensors for oxygen saturation, carbon dioxide concentration and other patient vitals may be relayed to a central monitoring station (e.g., at a hospital, ambulatory care center or central monitoring facilitating) to provide a remote patient with assistance, should it be required.

Other variations are possible. Therefore, it is intended that the scope of this disclosure include all aspects falling within the scope of the appended claims.

What is claimed is:

1. An intravascular gas exchange catheter comprising:
a catheter wall extending from a proximal end to a distal end, wherein the distal end comprises a first distal segment and a second distal segment;
an inflatable balloon structure disposed between the first distal segment and the second distal segment;
a first lumen port that is disposed at the proximal end and fluidly coupled to a first internal lumen adjacent the catheter wall; a second lumen port that is disposed at the proximal end and fluidly coupled to a second internal lumen that extends to a distal portion of the second distal segment; and a third lumen port that is disposed at the proximal end and fluidly coupled to an interior of the inflatable balloon structure by a third internal lumen;
wherein the first internal lumen and second internal lumen are fluidly isolated from each other along a length of the catheter, but fluidly coupled to each other at an interior space disposed at either the first distal segment or the second distal segment;
wherein the catheter wall comprises a material that facilitates diffusion of carbon dioxide from outside the catheter wall to the first interior lumen, and wherein a surface of the inflatable balloon structure is configured to facilitate passage of oxygen from inside the second internal lumen, through the surface, to a region outside the inflatable balloon structure.

2. The intravascular gas exchange catheter of claim 1, wherein the inflatable balloon structure comprises a plurality of petals, each petal having an interior space that is fluidly coupled to the first internal lumen.

3. The intravascular gas exchange catheter of claim 1, wherein the inflatable balloon structure comprises a plurality of wings, each wing having an interior space that is fluidly coupled to the first internal lumen.

4. The intravascular gas exchange catheter of claim 3, wherein each wing is configured to be collapsible onto the catheter wall when the intravascular gas exchange catheter is withdrawn into an introducer sheath.

5. The intravascular gas exchange catheter of claim 3, wherein a surface of each of the plurality of wings comprises a plurality of apertures, each aperture having a size of between 500 Angstroms and 4 um.

6. The intravascular gas exchange catheter of claim 5, wherein the apertures are configured to facilitate generation of microbubbles with diameters of 1-10 um when the intravascular gas exchange catheter is disposed in the vasculature of a patient and a supply of pressurized gas is applied to the first lumen port.

7. The intravascular gas exchange catheter of claim 1, further comprising an oxygen source fluidly coupled to the first internal lumen through an adjustable valve, a pressure sensor fluidly coupled to the first internal lumen, and a controller that receives as input a signal from the pressure sensor and outputs a control signal to the adjustable valve, the control signal causing the adjustable valve to close when an unexpected pressure drop is detected by the pressure sensor.

8. An intravascular gas exchange catheter comprising:
a catheter wall extending from a proximal end to a distal end;
a first internal lumen coupled to a first lumen port at the proximal end and adjacent at least a portion of the catheter wall, and a second internal lumen coupled to a second lumen port at the proximal end; and
an interior space enclosed by the catheter wall and disposed at the distal end, wherein the first internal lumen and second interior lumen are fluidly isolated from each other along a length of catheter wall but fluidly coupled to each other at the interior space;
wherein the catheter wall comprises a porous material that facilitates diffusion of a target gas through the catheter wall, from or to a space exterior to the catheter wall, to or from the first lumen.

9. The intravascular gas exchange catheter of claim 8, wherein the target gas is carbon dioxide.

10. An intravascular gas exchange catheter comprising:
a catheter wall extending from a proximal end to a distal end;
an inflatable balloon structure at the distal end; and
a lumen port at the proximal end and fluidly coupled to an internal lumen that is also fluidly coupled to an interior of the inflatable balloon structure; and
wherein a surface of the inflatable balloon structure comprises a plurality of apertures each having a diameter of between 500 Angstroms and 4 um.

11. The intravascular gas exchange catheter of claim 10, wherein the inflatable balloon structure comprises a plurality of petals or wings, each of which is configured to expand radially outward when pressure inside the internal lumen is positive and retract against the catheter wall when pressure inside the internal lumen is not positive.
